(19) United States
(12) Patent Application Publication
(10) Pub. No.: US 2026/0047747 A1
(43) Pub. Date: Feb. 19, 2026

(54) ENDOSCOPIC IMAGE PROCESSING APPARATUS AND METHOD FOR OPERATING ENDOSCOPIC IMAGE PROCESSING APPARATUS

(71) Applicant: OLYMPUS MEDICAL SYSTEMS CORP., Tokyo (JP)

(72) Inventor: Akihiro KUBOTA, Tokyo (JP)

(73) Assignee: OLYMPUS MEDICAL SYSTEMS CORP., Tokyo (JP)

(21) Appl. No.: 19/369,747

(22) Filed: Oct. 27, 2025

Related U.S. Application Data
(63) Continuation of application No. PCT/JP2023/016903, filed on Apr. 28, 2023.

(51) Int. Cl. A61B 1/00; A61B 1/273; A61B 1/31; G06T 7/00; G06V 10/25; G06V 10/70

(52) U.S. Cl. CPC A61B 1/000096; A61B 1/0002; A61B 1/00045; A61B 1/2736; A61B 1/31; G06T 7/0012; G06V 10/25; G06V 10/87; G06T 2207/10068; G06T 2207/20092; G06T 2207/30028; G06T 2207/30092; G06T 2207/30096; G06V 2201/031

(57) ABSTRACT

One or more processors select a model from among a plurality of types of machine learning models according to a region whose image is being picked up by an endoscope, generate notification information of a type of the model selected, receive an instruction signal for switching the model, measure a time interval from a selection of the model to a reception of the instruction signal, select a model selected immediately previously when the time interval is less than a first predetermined time, and select a model scheduled to be selected immediately subsequently when the time interval is equal to or greater than a second predetermined time.

### CROSS REFERENCE TO RELATED APPLICATION

This application is a continuation application of PCT/JP2023/016903 filed on Apr. 28, 2023, the entire contents of which are incorporated herein by this reference.

### FIELD

The present disclosure relates to an endoscopic image processing apparatus which selects and uses a machine learning model according to a region whose image is being picked up, from among a plurality of types of machine learning models, a method for operating the endoscopic image processing apparatus, and an endoscopic image processing program.

### BACKGROUND

In recent years, computer-aided detection (CADe), which indicates the position of a lesion candidate in a moving image picked up by an endoscope, and computer-aided diagnosis (CADx), which indicates discrimination information of the lesion candidate, have become known (CADe and CADx will be collectively referred to simply as CAD below). In CAD, for example, a machine-learning model that has undergone deep learning or the like using endoscopic images as training data is used.

Incidentally, when regions of a subject are organs, the color tone and lesion shape may differ from region to region. For this reason, if a single machine learning model is trained using images of various regions, there is a possibility that a trained model with low accuracy as CAD is generated.

Therefore, techniques have been proposed to train a machine learning model using images for respective regions of a subject, and generate a plurality of machine learning models that are respectively adapted to a plurality of regions. For example, a plurality of types of machine learning models, such as a machine learning model for the pharynx trained using only images of the pharynx, and a machine learning model for the esophagus trained using only images of the esophagus, are prepared in advance. Among the plurality of types of machine learning models, a machine learning model adapted to the image pickup region currently being observed with the endoscope can be used to increase the accuracy of the CAD.

For example, the publication of WO2022/181748 describes to provide, as CAD for endoscope, a plurality of machine learning models (lesion detectors) respectively corresponding to regions such as the pharynx, esophagus, stomach, and duodenum. The publication further describes that a device acquires information of the image pickup position and/or image pickup direction of a medical image, and that a selection unit selects a machine learning model according to the acquired information. This enables the user to alleviate the time and effort required for manually switching the machine learning model.

### SUMMARY

An endoscopic image processing apparatus according to one aspect of the present disclosure includes one or more processors. The one or more processors select a machine learning model from among a plurality of types of machine learning models according to a region whose image is being picked up by an endoscope, generate notification information of a type of the machine learning model selected, receive an instruction signal which is for switching the machine learning model and which is generated in response to a user's operation, measure a time interval from a selection of the machine learning model to a reception of the instruction signal, select a machine learning model selected immediately previously when the time interval is less than a first predetermined time, and select a machine learning model scheduled to be selected immediately subsequently when the time interval is equal to or greater than a second predetermined time that is the same as or longer than the first predetermined time.

In a method for operating an endoscopic image processing apparatus according to one aspect of the present disclosure, one or more processors select a machine learning model from among a plurality of types of machine learning models according to a region whose image is being picked up by an endoscope, generate notification information of a type of the machine learning model selected, receive an instruction signal for switching the machine learning model, the instruction signal being generated in response to a user's operation, measure a time interval from a selection of the machine learning model to a reception of the instruction signal, select a machine learning model selected immediately previously when the time interval is less than a first predetermined time, and select a machine learning model scheduled to be selected immediately subsequently when the time interval is equal to or greater than a second predetermined time that is the same as or longer than the first predetermined time.

A nonvolatile storage medium according to one aspect of the present disclosure stores an endoscopic image processing program. The program causes one or more computers to perform a process including selecting a machine learning model from among a plurality of types of machine learning models according to a region whose image is being picked up by an endoscope, generating notification information of a type of the machine learning model selected, receiving an instruction signal which is for switching the machine learning model and which is generated in response to a user's operation, measuring a time interval from a selection of the machine learning model to a reception of the instruction signal, selecting a machine learning model selected immediately previously when the time interval is less than a first predetermined time, and selecting a machine learning model scheduled to be selected immediately subsequently when the time interval is equal to or greater than a second predetermined time that is the same as or longer than the first predetermined time.

### BRIEF DESCRIPTION OF DRAWINGS

FIG. 1 is a diagram showing an example of a functional configuration of an endoscope system in a first embodiment of the present disclosure;

FIG. 2 is a block diagram showing an example of a functional configuration of a control section of the first embodiment;

FIG. 3 is a block diagram showing an example of a configuration in which an endoscopic image processing apparatus of the first embodiment includes a processor and a memory;

FIG. 4 is a flowchart showing an action of the endoscopic image processing apparatus of the first embodiment;

FIG. 5 is a diagram showing an example of display of a monitor in the endoscope system of the first embodiment;

FIG. 6 is a flowchart showing processing in an automatic model selection mode in step S3 of FIG. 4 in the first embodiment;

FIG. 7 is a flowchart showing an action of an endoscopic image processing apparatus of a second embodiment of the present disclosure;

FIG. 8 is a flowchart showing an action of an endoscopic image processing apparatus of a third embodiment of the present disclosure;

FIG. 9 is a block diagram showing a part of a functional configuration of an endoscopic image processing apparatus of a fourth embodiment of the present disclosure;

FIG. 10 is a flowchart showing an action of the endoscopic image processing apparatus of the fourth embodiment of the present disclosure;

FIG. 11 is a block diagram showing a part of a functional configuration of an endoscopic image processing apparatus of a fifth embodiment of the present disclosure;

FIG. 12 is a flowchart showing an action of the endoscopic image processing apparatus of the fifth embodiment of the present disclosure;

FIG. 13 is a flowchart showing an action of an endoscopic image processing apparatus of a sixth embodiment of the present disclosure;

FIG. 14 is a flowchart showing processing in a user selection mode in step S31 of FIG. 13 in the sixth embodiment;

FIG. 15 is a diagram showing how machine learning models are switched in turn in the user selection mode in the sixth embodiment;

FIG. 16 is a diagram showing an example of a functional configuration of an endoscope system in a seventh embodiment of the present disclosure;

FIG. 17 is a diagram showing a part of a configuration of an endoscope system in an eighth embodiment of the present disclosure;

FIG. 18 is a block diagram showing an example of a configuration of a lesion detection section when an endoscopic observation of the lower gastrointestinal tract is performed in a ninth embodiment of the present disclosure; and

FIG. 19 is a block diagram showing an example of a configuration in which a super-resolution processing section is provided in place of a lesion detection section in a tenth embodiment of the present disclosure.

### DESCRIPTION OF EMBODIMENTS

The following describes an embodiment of the present disclosure with reference to the drawings. However, the embodiments described below do not limit the present disclosure.

It is noted that, in the description of the drawings, the same or corresponding elements are marked with the same reference numerals as appropriate. It should also be noted that the drawings are schematic, and the relationship of lengths of elements, the ratio of lengths of elements, the quantity of elements, and the like within a single drawing may differ from the actual ones for the sake of simple description.

Furthermore, even among a plurality of drawings, they may include parts in which the mutual relations or proportions of the lengths differ from one another.

### First Embodiment

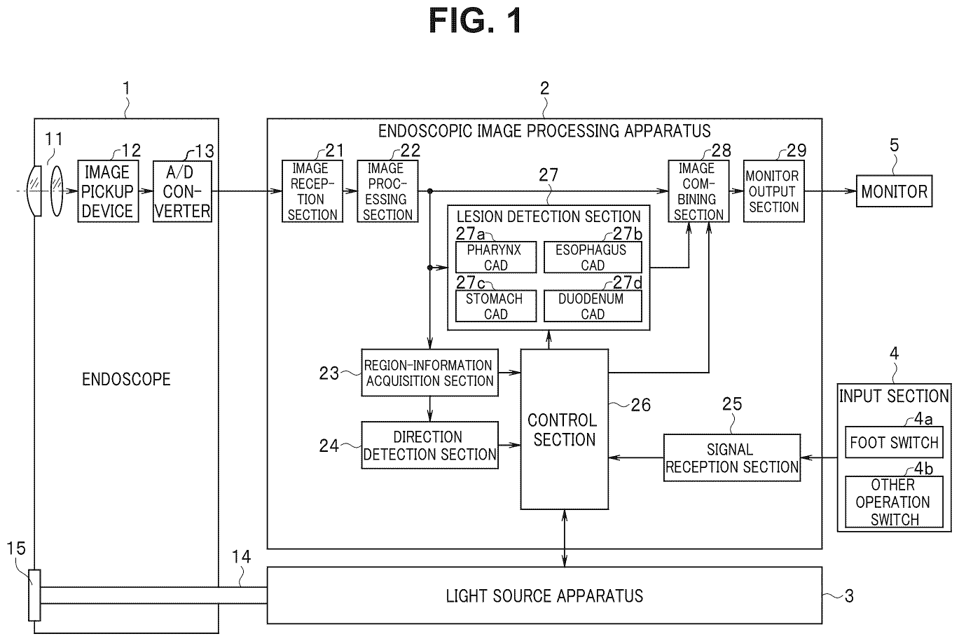

FIGS. 1 to 6 show the first embodiment of the present disclosure. FIG. 1 is a diagram showing an example of a functional configuration of an endoscope system in the first embodiment.

As shown in FIG. 1, the endoscope system includes an endoscope 1, an endoscopic image processing apparatus 2, a light source apparatus 3, an input section 4, and a monitor 5. The endoscope 1, the endoscopic image processing apparatus 2, the light source apparatus 3, the input section 4, and the monitor 5 are hardware components.

The endoscope 1 includes an image pickup lens 11, an image pickup device 12, an analog/digital (A/D) converter 13, a light guide 14, and an illumination lens 15.

The image pickup lens 11 includes one or more lenses, to form an optical image of a subject on the image pickup device 12. The optical image formed on the image pickup device 12 includes an image of one or more regions among a plurality of regions of the subject.

The image pickup device 12 photoelectrically converts (picks up) the optical image of the subject, to generate an analog image pickup signal pertaining to an endoscopic image. The image pickup lens 11 and the image pickup device 12 configure an image pickup system that picks up an image to acquire the endoscopic image.

The A/D converter 13 converts the analog image pickup signal to a digital image pickup signal. The endoscopic image pertaining to the digital image pickup signal is sent from the endoscope 1 to the endoscopic image processing apparatus 2. It is noted that an example has been shown here in which the A/D converter 13 is provided in the endoscope 1, but the A/D converter 13 can also be provided in the endoscopic image processing apparatus 2. The image pickup device 12 may be a digital image pickup device including the function of the A/D converter 13.

The light guide 14 transmits illumination light supplied from the light source apparatus 3 to the illumination lens 15.

The light source apparatus 3 may include, for example, a plurality of types of light sources corresponding to observation modes of the endoscope system. The observation modes include, for example, a normal light observation mode under which observation is performed using normal light (white light) and a special light observation mode under which observation is performed using special light.

Furthermore, the special light observation mode includes one or more of, for example, narrow-band imaging (NBI) mode, auto-fluorescence imaging (AFI) mode, infra-red imaging (IRI) mode, and the like. The light source apparatus 3 may include a system that changes the observation wavelength by means of a filter rather than a light source.

The illumination lens 15 irradiates the subject with the illumination light transmitted by the light guide 14. The illumination lens 15 and the image pickup system (the image pickup lens 11 and the image pickup device 12) are disposed at a distal end portion of an insertion portion of the endoscope 1.

The endoscopic image processing apparatus 2 may include, for example, an image reception section 21, an image processing section 22, a region-information acquisition section 23, a direction detection section 24, a signal reception section 25, a control section 26, a lesion detection section 27, an image combining section 28, and a monitor output section 29.

The image reception section 21 receives the endoscopic image from the endoscope 1.

The image processing section 22 performs image processing for image adjustment (image construction) on the endoscopic image received by the image reception section 21. The image processing performed by the image processing section 22 may include processing such as, for example, demosaicing, gain adjustment, white balance adjustment, gamma correction, noise reduction, contrast enhancement, and color change. Parameters of some of the processing performed by the image processing section 22, such as, for example, contrast enhancement and color change, may be user-settable.

The image processing section 22 sends the endoscopic image subjected to the image processing to the image combining section 28, the lesion detection section 27, and the region-information acquisition section 23. It is noted that the endoscopic image that is sent by the image processing section 22 to the lesion detection section 27 and the region-information acquisition section 23 does not have to be an endoscopic image subjected to all of the image processing. In other words, the image processing section 22 may send an endoscopic image before being subjected to the image processing or an endoscopic image subjected to a part of the image processing to the lesion detection section 27 and the region-information acquisition section 23.

The region-information acquisition section 23 acquires, in real time, information of a region whose image is being picked up by the endoscope 1 (current image-pickup-region information). The region-information acquisition section 23 may acquire the current image-pickup-region information based on the image, or can acquire the information by using an apparatus for position measurement, or the like. Examples of methods for acquiring the image-pickup-region information by the region-information acquisition section 23 will be described in embodiments mentioned below.

The direction detection section 24 detects a direction of travel of the endoscope 1. For example, when an endoscopy of the upper gastrointestinal tract is performed, the endoscope 1 is inserted from the oral cavity, and the distal end portion of the endoscope 1 is advanced from the pharynx to the esophagus and then to the stomach, and then reversed before and after reaching the duodenum, for example. After the reverse, the distal end portion of the endoscope 1 is withdrawn in an order of the stomach, esophagus, and pharynx.

The direction detection section 24 is capable of detecting the direction of travel of the distal end portion of the endoscope 1, based on, for example, the transition from the past to the present of the image-pickup-region information acquired by the region-information acquisition section 23, specifically, by comparing the image-pickup-region information immediately previous to the current and the current image-pickup-region information.

Specifically, when the immediately previous image-pickup-region information indicates the esophagus and the current image-pickup-region information indicates the stomach, the direction detection section 24 detects that the direction of travel is from the esophagus to the stomach. When the immediately previous image-pickup-region information indicates the stomach and the current image-pickup-region information indicates the esophagus, the direction detection section 24 detects that the direction of travel is from the stomach to the esophagus. Examples of other methods for detecting the direction of travel by the direction detection section 24 will be described in embodiments below.

The signal reception section 25 receives an instruction signal which is for switching the machine learning model, and which is generated by the input section 4 in response to the user's operation of the input section 4. Upon receiving the instruction signal, the signal reception section 25 sends a signal indicating reception of the instruction signal to the control section 26.

The input section 4 includes a switch or the like that can be operated by the user or an assistant operating the endoscope 1, to thereby send the instruction signal. In the example shown in FIG. 1, the input section 4 includes at least one of a foot switch 4a or another operation switch 4b other than the foot switch 4a.

The foot switch 4a is, for example, an external switch connected to the endoscopic image processing apparatus 2, and is disposed at a position where the user operating the endoscope 1 can operate with the foot. The user is capable of sending the instruction signal by, for example, simply performing an operation to step on the foot switch 4a once.

The other operation switch 4b may be an operation switch to be actuated with a button that is provided, for example, on an operation portion of the endoscope 1.

The user is capable of sending the instruction signal by, for example, simply pressing once the button that operates the other operation switch 4b.

Furthermore, the other operation switch 4b may be an operation switch or the like that is actuated based on voice recognition.

As mentioned below, switching of the machine learning model by the instruction signal is performed according to the timing at which the instruction signal is received. For this reason, the instruction signal does not need to include information such as indicating which type of machine learning model the user requests to switch to, and may be a simple timing signal. Thus, the user is capable of sending the instruction signal from the input section 4 with an easy operation.

The control section 26 also serves as a system controller that controls the entire endoscope system. For example, the control section 26 controls the light source apparatus 3, setting the type of a light source according to the observation mode, and instructing the amount of light emission. The control section 26 generates light source information which is based on the control of the light source apparatus. The light source information includes, for example, information of whether the type of the current light source is a light source emitting normal light or a light source emitting special light.

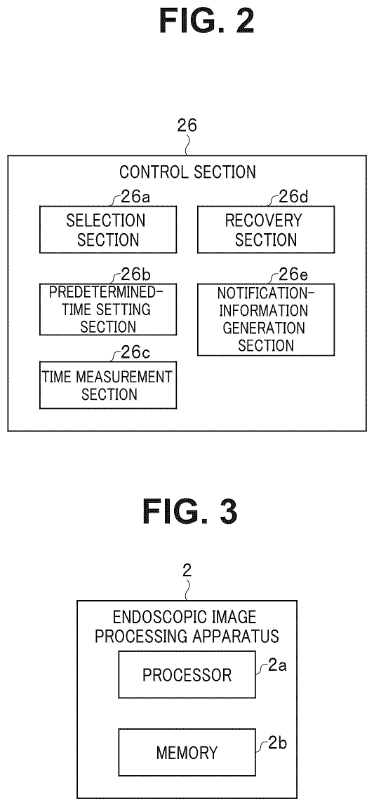

FIG. 2 is a block diagram showing an example of a functional configuration of the control section 26 of the first embodiment.

The control section 26 includes a selection section 26a, a predetermined-time setting section 26b, a time measurement section 26c, a recovery section 26d, and a notification-information generation section 26e.

The selection section 26a selects a machine learning model according to the region whose image is being picked up by the endoscope 1, from among a plurality of types of machine learning models that are respectively adapted to the plurality of regions arranged in the subject. For the sake of simplicity, “machine learning model” may be simply referred to as “model.

Here, the present embodiment shows an example of including the lesion detection section 27 which performs, as the machine learning model, computer-aided detection (CADe) which indicates the position of a lesion candidate, and/or computer-aided diagnosis (CADx) which indicates discrimination information of the lesion candidate (as mentioned above, CADe and CADx are collectively referred to simply as CAD).

Furthermore, the present embodiment assumes an endoscope system for examining the upper gastrointestinal tract, and the plurality of regions of the subject are, for example, organs including the pharynx, esophagus, stomach, and duodenum, which are arranged in order in the subject.

In this case, the lesion detection section 27 includes, as the plurality of types of machine learning models, a machine learning model 27a for the pharynx (pharynx CAD), a machine learning model 27b for the esophagus (esophagus CAD), a machine learning model 27c for the stomach (stomach CAD), and a machine learning model 27d for the duodenum (duodenum CAD), which are respectively adapted to the organs of the pharynx, esophagus, stomach, and duodenum.

The machine learning model 27a for the pharynx is a trained model that is trained for CAD using images of the pharynx as training data. The machine learning model 27b for the esophagus is a trained model that is trained for CAD using images of the esophagus as training data. The machine learning model 27c for the stomach is a trained model that is trained for CAD using images of the stomach as training data. The machine learning model 27d for the duodenum is a trained model that is trained for CAD using images of the duodenum as training data.

As the machine learning model, for example, a deep neural network (DNN) that includes a plurality of hidden layers and performs deep learning may be used. However, the machine learning model is not limited to this, but a known model such as convolution neural networks (CNN), regions with CNN features (R-CNN) utilizing CNN, or fully convolutional networks (FCN) can be appropriately used as the machine learning model.

It is noted that, the above showed an example in which the upper gastrointestinal tract is divided into four regions in organ units, and models (lesion detectors) for the respective regions are used. However, each region may be further divided into a plurality of regions, and models for the respective divided regions can be used. For example, the esophagus can be divided into the upper esophagus and the lower esophagus (or the upper esophagus and the middle and lower esophagus), and a machine learning model for the upper esophagus and a machine learning model for the lower esophagus (or the middle and lower esophagus) can be used, respectively.

A plurality of models corresponding to types of light sources may be provided for the same region. In other words, for example, a model for normal light and a model for special light may be provided for each of the plurality of regions, and which one of the models to use may be selected according to the light source information.

The selection section 26a selects a machine learning model to be used by the lesion detection section 27 from among the plurality of types of machine learning models 27a to 27d, according to the current image-pickup-region information by the endoscope 1 acquired by the region-information acquisition section 23.

As mentioned above, the pharynx, esophagus, stomach, and duodenum of the upper gastrointestinal tract are arranged in this order within the subject. Then, if the direction of travel is detected by the direction detection section 24, a switching order of the plurality of regions is defined. This allows the selection section 26a to schedule, according to the switching order, a machine learning model to be selected immediately subsequently.

In this manner, the selection section 26a selects a machine learning model from among the plurality of types of machine learning models 27a to 27d according to the current image-pickup-region information acquired by the region-information acquisition section 23, based on the switching order defined according to the arrangement of the plurality of regions and on the direction of travel which is detected by the direction detection section 24.

When the light source apparatus 3 includes a plurality of types of light sources corresponding to a plurality of observation modes, the selection section 26a may select, further according to the light source information, a machine learning model to be used by the lesion detection section 27.

The predetermined-time setting section 26b sets a first predetermined time and a second predetermined time. The second predetermined time is the same as or longer than the first predetermined time. It is noted that the present embodiment describes an example in which the first and second predetermined times are the same predetermined time, and an example in which the second predetermined time is longer than the first predetermined time will be described in embodiment mentioned below.

A predetermined time (hereinafter including the first predetermined time and the second predetermined time) is a threshold time to be compared to the time interval from the time point when the selection section 26a selects the machine learning model to the time point when the signal reception section 25 receives the instruction signal sent from the input section 4.

The predetermined time may be a fixed amount of time that is independent of the type of the machine learning model selected and the direction of travel of the endoscope 1. The predetermined time can also be made different depending on the direction of travel of the endoscope 1 (e.g., a direction from the esophagus to the stomach or a direction from the stomach to the esophagus). Furthermore, the predetermined time may be made different depending on the type of the machine learning model selected.

An example in which the predetermined time when the selection section 26a selects a machine learning model adapted to a first region is made different from the predetermined time when the selection section 26a selects a machine learning model adapted to another region than the first region is as follows, for example.

The predetermined time when the selection section 26a selects the machine learning model 27c for the stomach is longer than the predetermined time when the selection section 26a selects the machine learning model 27a for the pharynx, and longer than the predetermined time when the selection section 26a selects the machine learning model 27d for the duodenum.

Furthermore, the predetermined time when the selection section 26a selects the machine learning model 27b for the esophagus is longer than the predetermined time when the selection section 26a selects the machine learning model 27a for the pharynx, and longer than the predetermined time when the selection section 26a selects the machine learning model 27d for the duodenum.

It is noted that one region may be observed separately depending on the direction of travel. For example, when the direction of travel of the endoscope 1 is, for example, from the pharynx to the esophagus (in the insertion case), the upper part of the esophagus is observed, and when the direction of travel of the endoscope 1 is, for example, from the stomach to the esophagus (in the withdrawal case), the lower and middle parts of the esophagus are observed. In this case, the predetermined time for observing the upper esophagus during insertion and the predetermined time for observing the lower and middle esophagus during withdrawal may each be shorter than the predetermined time for observing the stomach, longer than the predetermined time for observing the pharynx and duodenum, or the like.

Furthermore, the predetermined time for observing the duodenum may be almost the same as the predetermined time for observing the pharynx, or can be somewhat longer than the predetermined time for observing the pharynx.

When there are a plurality of switches for the user to send an instruction signal, the predetermined time may be made different for each switch. For example, as shown in FIG. 1, suppose that the switches for the user to send the instruction signal in the input section 4 include at least one of the foot switch 4a or the other operation switch 4b other than the foot switch 4a. When the foot switch 4a generates a first instruction signal and the other operation switch 4b generates a second instruction signal, the predetermined time that is set for a case where the signal reception section 25 receives the first instruction signal may be longer than the predetermined time that is set for a case where the signal reception section 25 receives the second instruction signal. The operation of the input section 4 or the action on the input section 4 by the user may be preferably two or fewer actions, and may more preferably be one action. Examples of the one action include stepping on a foot pedal, sliding the foot pedal, pressing a button provided on the operation portion of the endoscope, moving the button provided on the operation portion of the endoscope, and uttering a predetermined word.

Furthermore, the predetermined time may be set by the user as desired.

The time measurement section 26c measures the time interval from when the selection section 26a selects the machine learning model to when the signal reception section 25 receives the instruction signal. It is noted that, when a time lag of a predetermined time or more occurs from when the region-information acquisition section 23 recognizes a region different from one that it had been recognized until immediately before to when the selection section 26a selects a machine learning model, measurement of the time interval may begin from the time point when the region-information acquisition section 23 recognizes the different region from the one that it had been recognized until immediately before.

When the time interval measured by the time measurement section 26c is less than the predetermined time, the recovery section 26d causes the selection section 26a to select a machine learning model that was selected immediately previously by the selection section 26a.

When the time interval measured by the time measurement section 26c is equal to or greater than the predetermined time, the recovery section 26d causes the selection section 26a to select a machine learning model scheduled to be selected immediately subsequently by the selection section 26a.

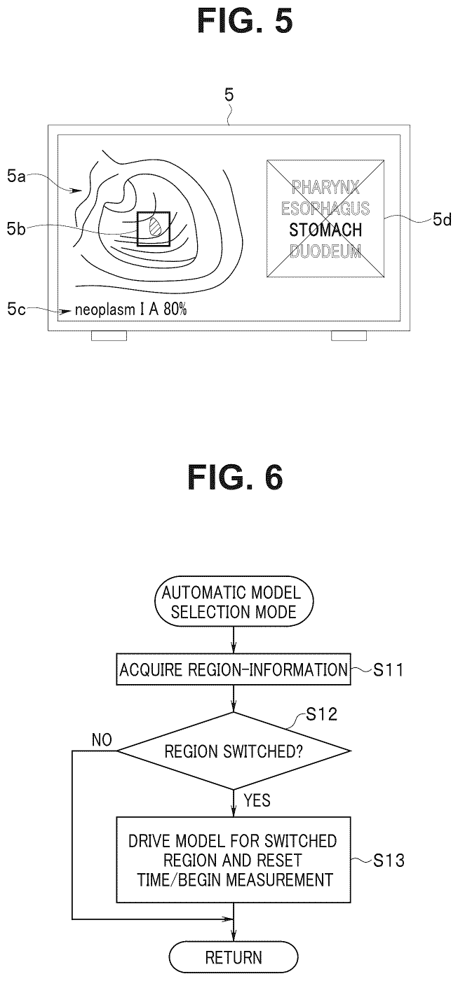

The notification-information generation section 26e generates notification information of the type of the machine learning model selected by the selection section 26a, and sends the notification information to the image combining section 28. It is noted that an example in which the notification information is information displayed on the monitor 5 is shown in FIG. 5 below, but the notification information is not limited thereto, and may also be audio information, or the like.

The machine learning model of the lesion detection section 27, which was selected by the selection section 26a, receives input of an endoscopic image from the image processing section 22 to perform inference, and sends an inference result to the image combining section 28. When the machine learning model includes CADe and CADx functions, the inference result includes position information of the lesion candidate and discrimination information of the lesion candidate.

The image combining section 28 combines, into a single image, the endoscopic image received from the image processing section 22, CAD information such as a lesion detection result received from the lesion detection section 27, and the type (name) of the machine learning model (lesion detector) that is currently selected by the selection section 26a, and was received from the notification-information generation section 26e.

The monitor output section 29 outputs the image combined by the image combining section 28 to the monitor 5. Thus, the monitor output section 29 outputs, to the monitor 5, an image including the endoscopic image and the inference result by the machine learning model.

FIG. 3 is a block diagram showing an example of a configuration in which the endoscopic image processing apparatus 2 of the first embodiment includes a processor 2a and a memory 2b.

The endoscopic image processing apparatus 2 includes the processor 2a and the memory 2b, for example. The processor 2a and the memory 2b are hardware components. The processor 2a is configured by an application specific integrated circuit (ASIC) including a central processing unit (CPU), and the like, a field programmable gate array (FPGA), and the like.

The memory 2b is a storage medium that stores, in a nonvolatile manner, a processing program that causes the processor 2a to implement respective functions of circuits. By reading and executing the processing program stored in the memory 2b, the processor 2a operates as at least a part of functional sections of the endoscopic image processing apparatus 2 shown in FIGS. 1 and 2. The endoscopic image processing apparatus 2 executes the processing program (endoscopic image processing program), to thereby implement a method for operating the endoscopic image processing apparatus and an endoscopic image processing method. The endoscopic image processing apparatus 2 may include a plurality of processors 2a.

However, a part or all of the functional sections of the endoscopic image processing apparatus 2 shown in FIGS. 1 and 2 may be configured by a dedicated electronic circuit.

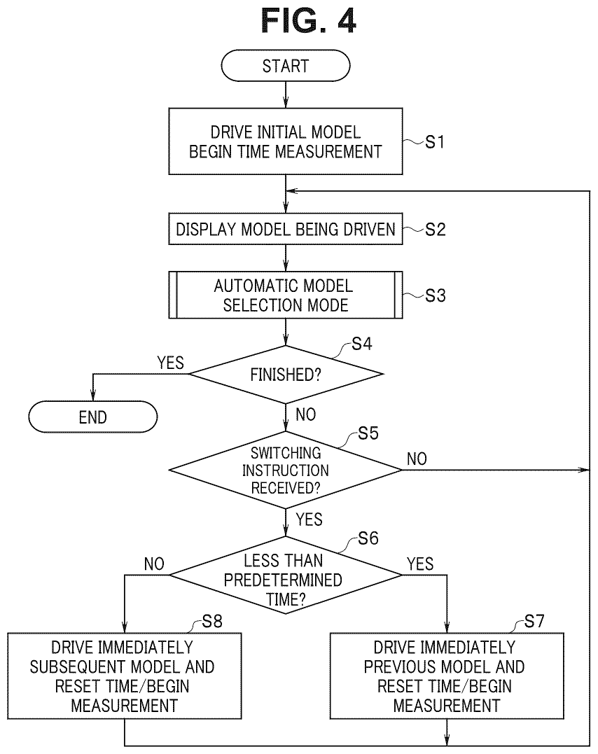

FIG. 4 is a flowchart showing an action of the endoscopic image processing apparatus 2 of the first embodiment.

For example, when the endoscopy of the upper gastrointestinal tract begins, the endoscope 1 is inserted from the oral cavity and enters the pharynx. Then, the selection section 26a selects and drives the machine learning model 27a for the pharynx that should be selected initially, among the machine learning models 27a to 27d included in the lesion detection section 27, to cause the machine learning model 27a to perform inference. Furthermore, the time measurement section 26c begins measuring the time from the time point when the machine learning model 27a for the pharynx is selected (step S1).

The endoscopic image processing apparatus 2 outputs a composite image to the monitor 5, to cause the monitor 5 to display the type of the machine learning model being driven (step S2).

Here, FIG. 5 is a diagram showing an example of display of the monitor 5 in the endoscope system of the first embodiment.

On the monitor 5, an endoscopic image 5a, an icon 5b indicating the position of a lesion candidate in the endoscopic image 5a, information 5c of a discrimination result and a progression assessment of the lesion candidate indicated by the icon 5b, and a type 5d of the machine learning model that is currently selected (driven) are displayed, as a result of being combined into one image by the image combining section 28.

Of these, the icon 5b and the information 5c on the discrimination result and the progression assessment are combined into the one image by the image combining section 28, based on the inference result (CAD information) by any of the machine learning models 27a to 27d. The type 5d of the machine learning model is combined into the one image by the image combining section 28, based on the notification information of the type of the machine learning model received from the notification-information generation section 26e.

In the example shown in FIG. 5, all types of the machine learning models 27a to 27d included in the lesion detection section 27 are displayed in the display section of the type 5d of the machine learning model. In the present embodiment, the names of the regions to which the machine learning models 27a to 27d are adapted are used as the types of the machine learning models 27a to 27d. The type of the machine learning model that is currently selected among all the types is displayed distinguishably from the types of unselected machine learning models.

Specific examples of the distinguishable display include, for example, displaying the types of unselected machine learning models with low saturation such as light gray, and displaying the type of the selected machine learning model with black or high saturation such as a bright primary color. Only the selected machine learning model may be highlight-displayed. Display methods are not limited to these, but other distinguishable and appropriate ones can be used.

Furthermore, the endoscopic image processing apparatus 2 executes the processing in the automatic model selection mode (step S3). FIG. 6 is a flowchart showing the processing in the automatic model selection mode in step S3 of FIG. 4 in the first embodiment.

When the processing in the automatic model selection mode is entered, the selection section 26a acquires the current image-pickup-region information from the region-information acquisition section 23 (step S11).

The selection section 26a determines whether the region to which the currently selected machine learning model is adapted is different from the current image pickup region indicated by the information acquired from the region-information acquisition section 23 (i.e., whether or not the current image pickup region has switched) (step S12).

If it is determined that the current image pickup region has switched, the selection section 26a drives the machine learning model adapted to the current image pickup region indicated by the information acquired from the region-information acquisition section 23, and the time measurement section 26c resets the time being measured and begins measuring the time again (step S13).

If the processing in step S13 is performed, or if it is determined in step S12 that the current image pickup region has not switched, the processing returns to the processing in FIG. 4.

If the processing shown in FIG. 6 returns to the processing shown in FIG. 4, the control section 26 determines whether or not to finish the endoscopic observation (step S4).

If it is determined that the endoscopic observation is not to be finished, the recovery section 26d determines whether or not the signal reception section 25 has received the instruction signal for switching the machine learning model (step S5).

If it is determined that the signal reception section 25 has not received the instruction signal, the processing returns to step S2 and a series of processing for automatically selecting a model is continued while the model being driven is displayed.

On the other hand, if it is determined in step S5 that the signal reception section 25 has received the instruction signal, the recovery section 26d determines whether the time being measured by the time measurement section 26c (the time interval from when the selection section 26a selects the current machine learning model until the signal reception section 25 receives the instruction signal) is less than the predetermined time or equal to or greater than the predetermined time (step S6).

If it is determined that the time is less than the predetermined time, the recovery section 26d causes the selection section 26a to select and drive the machine learning model that was selected immediately previously by the selection section 26a. Furthermore, the time measurement section 26c resets the time being measured and begins measuring the time again (step S7).

In other words, suppose that a switch has been made to the current machine learning model not matching the user's needs, even though the machine learning model matching the user's needs is one that the selection section 26a selected immediately previously. In this case, the user views the type 5d of the machine learning model currently selected, which is displayed on the monitor 5, and wishes to immediately switch to the machine learning model matching the user's needs. Thus, the machine learning model can match the user's needs by switching the machine learning model to the one selected immediately previously when the time interval is less than the predetermined time.

If it is determined that the time is equal to or greater than the predetermined time, the recovery section 26d causes the selection section 26a to select and drive the machine learning model that the selection section 26a is scheduled to select immediately subsequently according to the switching order. Furthermore, the time measurement section 26c resets the time being measured and begins measuring the time again (step S8).

In other words, it is when the endoscope has moved through the current image pickup region and approaches the boundary with the next region that the machine learning model matching the user's needs becomes the machine learning model that the selection section 26a is scheduled to select immediately subsequently. It takes a fixed amount of time or more before the observation of the current image pickup region is almost ended and the next region is approached. Therefore, if the instruction signal is received when the time interval is equal to or greater than the predetermined time, it can be estimated that the machine learning model scheduled to be selected immediately subsequently matches the user's needs. In this manner, by switching to the machine learning model scheduled to be selected immediately subsequently when the time interval is equal to or greater than the predetermined time, the machine learning model can be made to match the user's needs.

After the processing in step S7 or step S8 is performed, the processing returns to step S2 and the processing mentioned above is performed.

Thereafter, if the control section 26 determines in step S4 that the endoscopic observation is to be finished, the endoscopic image processing apparatus 2 ends the series of processing shown in FIG. 4.

According to the first embodiment, when the automatically selected machine learning model does not match the user's needs, the user is capable of selecting a machine learning model matching the user's needs with a simple operation such as pressing a button once.

The user does not need to perform complex operations to select a machine learning model. This enables to suppress a decrease in the labor-saving effectiveness (the effect of saving user's labor) of the endoscopic image processing apparatus 2 having the function of automatically selecting a machine learning model. This allows the user to concentrate on operating the endoscope 1 and checking the display on the monitor 5, thereby improving the efficiency of endoscopy.

It is noted that, when the endoscope 1 is for medical use, the user is assumed to be a human doctor, but is not limited thereto. For example, the user may be an artificial intelligence (AI) doctor.

### Second Embodiment

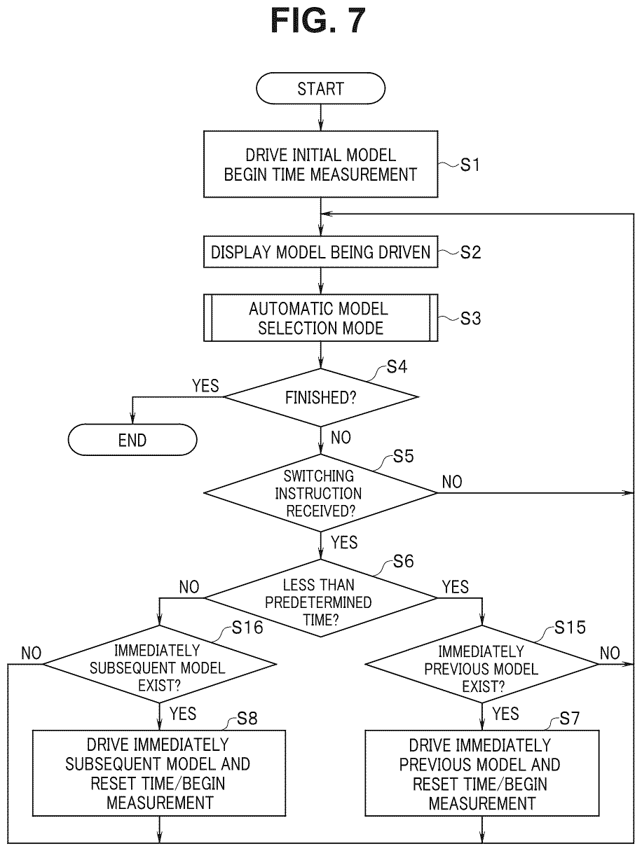

FIG. 7 is a flowchart showing an action of the endoscopic image processing apparatus 2 of the second embodiment of the present disclosure. In the second embodiment, parts similar to those of the first embodiment are marked with the same reference numerals, and descriptions thereof will be omitted as appropriate. In the second embodiment, points different from the first embodiment will be mainly described.

In the endoscopy to observe the upper gastrointestinal tract, the lesion detection section 27 includes the machine learning model 27a for the pharynx, the machine learning model 27b for the esophagus, the machine learning model 27c for the stomach, and the machine learning model 27d for the duodenum, as shown in FIG. 1, for example.

When the endoscopy begins and the machine learning model 27a for the pharynx is selected as the initial model in step S1, even if a switching instruction is received in step S6 within less than the predetermined time, the immediately previous model that should be switched to in step S7, that is, a machine learning model adapted to a region further along the withdrawal direction from the pharynx, does not exist. In such a case, it can be determined that the signal reception section 25 received the switching instruction signal as a result of an erroneous button operation by the user.

Similarly, even if a switching instruction is received after a predetermined time or more has elapsed in step S6 since the selection section 26a selected the machine learning model 27d for the duodenum, there is no immediately subsequent model to be switched to in step S8, i.e., no machine learning model adapted to a region further along the insertion direction from the duodenum. In such a case, it can be determined that the signal reception section 25 received the switching instruction signal as a result of an erroneous button operation by the user.

The endoscopic image processing apparatus 2 of the present embodiment performs processing to deal with such an error in the user's operation.

That is, in FIG. 7, if it is determined in step S6 that the time is less than the predetermined time, it is determined whether or not an immediately previous machine learning model in the direction of travel of the endoscope 1 exists (step S15). If it is determined here that the immediately previous machine learning model exists, the processing proceeds to the processing in step S7.

On the other hand, if it is determined in step S15 that the immediately previous machine learning model does not exist, it is determined that the user made an erroneous button operation and the processing returns to the processing in step S2 without switching the machine learning model.

If it is determined in step S6 that the time is equal to or greater than the predetermined time, it is determined whether or not an immediately subsequent machine learning model in the direction of travel of the endoscope 1 exists (step S16). If it is determined here that the immediately subsequent machine learning model exists, the processing proceeds to the processing in step S8.

On the other hand, if it is determined in step S16 that the immediately subsequent machine learning model does not exist, it is determined that the user made an erroneous button operation and the processing returns to the processing in step S2 without switching the machine learning model.

The second embodiment yields an effect that is almost the same as that of the first embodiment mentioned above.

According to the second embodiment, even if the user erroneously operates to perform an operation to select a machine learning model that does not exist, the selection of an appropriate machine learning model can be maintained without being affected by the erroneous operation.

It is noted that the processing shown in steps S15 and S16 of FIG. 7 may be appropriately applied to each embodiment mentioned below.

### Third Embodiment

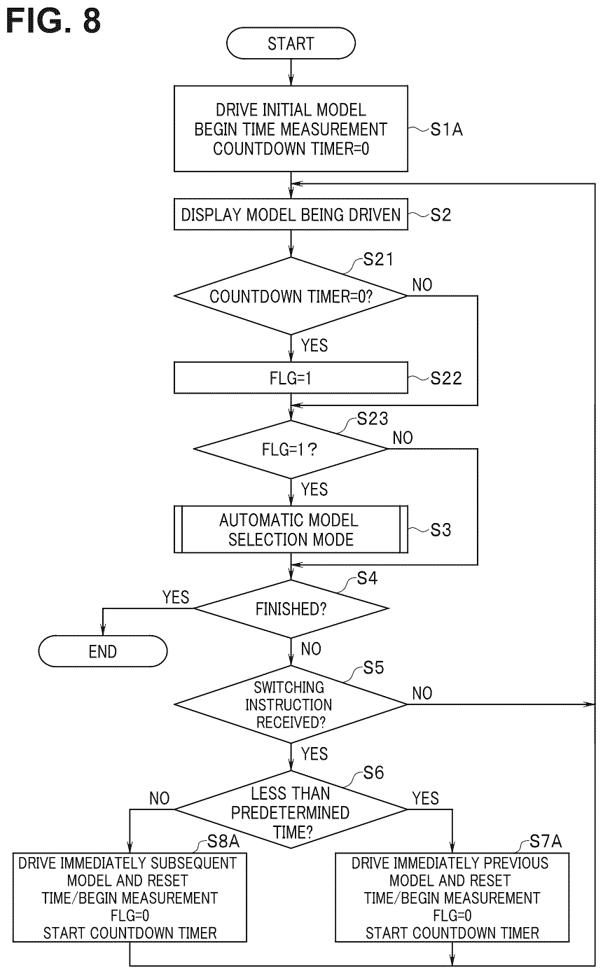

FIG. 8 is a flowchart showing an action of the endoscopic image processing apparatus 2 of the third embodiment of the present disclosure. In the third embodiment, parts similar to those of the first and second embodiments are marked with the same reference numerals, and descriptions thereof will be omitted as appropriate. In the third embodiment, points different from the first and second embodiments will be mainly described.

In the processing shown in FIG. 4, after switching the machine learning model in step S7 or step S8, the processing returns to the processing in step S2 and step S3. In this case, there is a possibility that the machine learning model that was switched to in step S7 or step S8 in response to the user's switching instruction performs the processing in the automatic model selection mode in step S3, resulting in reverting to the machine learning model automatically selected according to the current region information.

Therefore, the present embodiment is designed so that the machine learning model that is switched to in step S7A or step S8A in response to the user's switching instruction continues to be selected for a fixed amount of time (third predetermined time). To this effect, the present embodiment includes a countdown timer to count down the fixed amount of time, and a flag FLG to indicate whether or not to execute the automatic model selection mode. The flag FLG takes a value of “1” or “0”. Flag FLG=1 indicates that the automatic model selection mode is to be executed. Flag FLG=0 indicates that the automatic model selection mode is not to be executed.

When the processing in FIG. 8 begins, the selection section 26a selects and drives the initial model, the time measurement section 26c begins measuring the time since the model was selected, and the recovery section 26d sets the countdown timer to 0 (step S1A).

Thereafter, after the processing in step S2 is performed, the recovery section 26d determines whether or not the countdown timer is 0 (step S21).

If the endoscopy begins and the processing in step S1A is performed, it is determined in step S21 that the countdown timer is 0. At this time, the recovery section 26d sets the flag FLG to 1 (step S22).

If step S22 is performed or if it is determined in step S21 that the countdown timer is not 0, the recovery section 26d determines whether or not the flag FLG is 1 (step S23).

Here, if it is determined that the flag FLG is 1, the selection section 26a performs the processing in the automatic model selection mode in step S3. If it is determined that the flag FLG is 0, the selection section 26a skips the processing in step S3.

Thereafter, the processing in steps S4 to S6 are performed, and if it is determined in step S6 that the time is less than the predetermined time, the selection section 26a selects and drives the machine learning model that it selected immediately previously, the time measurement section 26c resets the measurement time to begin the time measurement, and the recovery section 26d sets the flag FLG to 0 and starts the countdown timer for a fixed amount of time (step S7A).

If it is determined in step S6 that the time is equal to or greater than the predetermined time, the selection section 26a selects and drives the machine learning model that it is scheduled to select immediately subsequently, the time measurement section 26c resets the measurement time to begin the time measurement, and the recovery section 26d sets the flag FLG to 0 to start the countdown timer for the fixed amount of time (step S8A).

After the processing in step S7A or step S8A are performed, the processing returns to the processing in step S2.

Then, the flag FLG set to 0 in step S7A or step S8A is not set to 1 in step S22 until the countdown timer is determined to have reached 0 in step S21. Therefore, until the countdown timer reaches 0, the flag FLG is maintained at 0, and the processing in the automatic model selection mode in step S3 is skipped by the determination in step S23.

The third embodiment yields an effect that is almost the same as those of the first and second embodiments mentioned above.

According to the third embodiment, the recovery section 26d prohibits the automatic selection of the machine learning model according to the current image-pickup-region information by the selection section 26a for the fixed amount of time (third predetermined time) after the machine learning model is switched in response to the instruction signal. This prevents the machine learning model that has been switched in response to the instruction signal from immediately reverting to the original machine learning model due to the processing in the automatic model selection mode, allowing to perform processing that respects the user's switching instructions.

It is noted that the processing of prohibiting the automatic selection of the machine learning model for the third predetermined time shown in FIG. 8 may be appropriately applied to each embodiment mentioned below.

### Fourth Embodiment

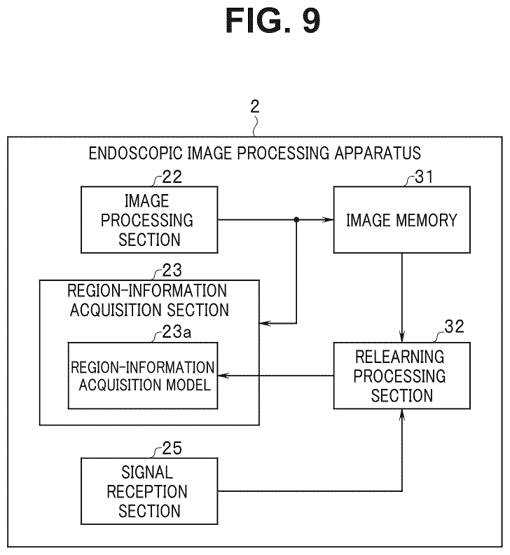

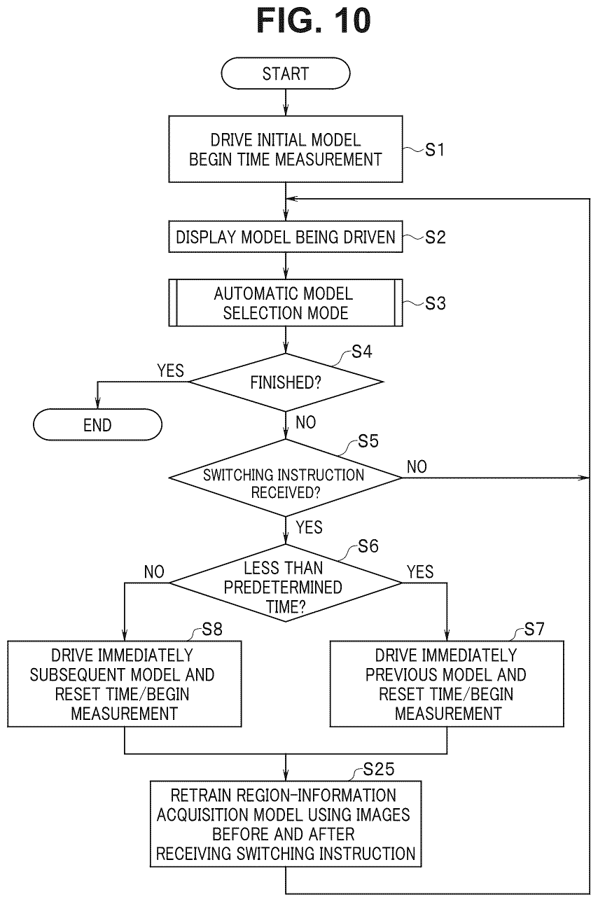

FIGS. 9 and 10 show the fourth embodiment of the present disclosure. FIG. 9 is a block diagram showing a part of a functional configuration of the endoscopic image processing apparatus 2 of the fourth embodiment. In FIG. 9, several illustrations of the configuration shown in FIG. 1 are omitted.

In the fourth embodiment, parts similar to those of the first to third embodiments are marked with the same reference numerals, and descriptions thereof will be omitted as appropriate. In the fourth embodiment, points different from the first to third embodiments will be mainly described.

The fact that the user performs an operation to send an instruction signal from the input section 4 means that the automatic selection of the machine learning model in step S13 based on the current image-pickup-region information acquired in step S11 of FIG. 6 does not match the user's needs.

Therefore, the present embodiment is configured to improve the performance that the region-information acquisition section 23 acquires the current image-pickup-region information.

The region-information acquisition section 23 includes a second machine learning model 23a that acquires region information (which is a region-information acquisition model, i.e., a second machine learning model different from the machine learning models 27a to 27d of the lesion detection section 27). The second machine learning model 23a receives input of an endoscopic image from the image processing section 22, performs inference, and outputs the current image-pickup-region information as an inference result.

It is noted that, in FIG. 9, for simplicity of description, an example is shown in which the region-information acquisition section 23 includes one second machine learning model 23a. However, no limitation is placed on this example, and models for respective regions may be used each as the second machine learning model 23a in order to improve the accuracy of inference.

As a specific example, for the endoscopy that observes the upper gastrointestinal tract, a machine learning model for acquiring region information of the pharynx, a machine learning model for acquiring region information of the esophagus, a machine learning model for acquiring region information of the stomach, and a machine learning model for acquiring region information of the duodenum are provided in the region-information acquisition section 23. The endoscopic image that the region-information acquisition section 23 receives from the image processing section 22 is inputted into each of the machine learning models.

The machine learning model for acquiring the region information of the pharynx outputs a score that the inputted endoscopic image is an endoscopic image of the region of the pharynx. The machine learning model for acquiring the region information of the esophagus outputs a score that the inputted endoscopic image is an endoscopic image of the region of the esophagus. The machine learning model for acquiring the region information of the stomach outputs a score that the inputted endoscopic image is an endoscopic image of the region of the stomach. The machine learning model for acquiring the region information of the duodenum outputs a score that the inputted endoscopic image is an endoscopic image of the region of the duodenum.

The region-information acquisition section 23 outputs, as the current image-pickup-region information, information of the region corresponding to the machine learning model that has output the highest score.

In addition to the configuration shown in FIG. 1, the endoscopic image processing apparatus 2 of the present embodiment includes an image memory 31 and a relearning processing section 32. The image memory 31 is a hardware component. The processor 2a in FIG. 3 operates as the relearning processing section 32 by reading and executing a processing program stored in the memory 2b.

The image memory 31 is connected to the image processing section 22. The image memory 31 stores an endoscopic image same as that output from the image processing section 22 to the region-information acquisition section 23. The image memory 31 may store all the inputted endoscopic images.

However, what is needed for the retraining described in the present embodiment are endoscopic images for several frames before and after the time point at which the instruction signal is received. Therefore, the image memory 31 may reduce the memory capacity by storing endoscopic images for one or more frames before and after the time point at which the instruction signal is received.

The relearning processing section 32 is connected to the image memory 31, the signal reception section 25, and the second machine learning model 23a.

FIG. 10 is a flowchart showing an action of the endoscopic image processing apparatus 2 of the fourth embodiment.

In the processing shown in FIG. 10, processing in steps S1 to S8, similar to those in FIG. 4, are performed.

Suppose that, at this time, in step S5, the signal reception section 25 receives the instruction signal for switching the machine learning models 27a to 27d.

Then, after the processing in step S7 or S8 is performed, the relearning processing section 32 reads out, from the image memory 31, endoscopic images for several frames before and after the time point at which the signal reception section 25 received the instruction signal (including the endoscopic image pertaining to the image that the monitor output section 29 outputs to the monitor 5 when the instruction signal was received), and uses the read-out endoscopic images for several frames as training data to retrain the second machine learning model 23a (step S25).

In the retraining, correct answer information is that an endoscopic image, which is the training data used by the second machine learning model 23a, is the image of the region to which a machine learning model of the lesion detection section 27 switched to by the instruction signal is adapted. This allows the retrained second machine learning model 23a to infer the current image-pickup-region information that matches the user's needs.

In a case where a model is used for each region, a specific example is as follows. Suppose that, as a result of reception of the instruction signal in step S5, the model is switched to the machine learning model 27c for the stomach, for example, in step S7 or step S8.

In this case, what is correct as the current image pickup region is the stomach. Therefore, endoscopic images for several frames before and after the time point at which the instruction signal is received are used as correct answer images to retrain the machine learning model that acquires the region information of the stomach, and are used as incorrect answer images to retrain each of the machine learning models that acquire the region information of the pharynx, esophagus, and duodenum, respectively. In this way, the retrained region-information acquisition section 23 can infer, with higher accuracy, that the current image pickup region is the stomach.

### After the Processing in Step S25 Is Performed, the Processing Returns to the

processing in step S2.

It is noted that, FIGS. 9 and 10 describes an example of performing real-time retraining within the endoscopic image processing apparatus 2, the present disclosure is not limited to this example. For example, the second machine learning model 23a may be retrained after the endoscopy is finished. In this case, the image memory 31 only has to store, in a nonvolatile manner, endoscopic images for several frames before and after the time point of reception each time the signal reception section 25 receives the instruction signal. The retraining after the endoscopy is finished is not limited to being performed within the endoscopic image processing apparatus 2. A separate machine learning apparatus may be used to retrain the second machine learning model 23a, and the retrained second machine learning model 23a may be returned to the region-information acquisition section 23.

The fourth embodiment yields an effect that is almost the same as those of the first to third embodiments mentioned above.

According to the fourth embodiment, when the signal reception section 25 receives the instruction signal, the endoscopic image pertaining to the image that is outputted to the monitor 5 when receiving the instruction signal (i.e., the endoscopic image pertaining to the image that the user is observing when sending the instruction signal) (the endoscopic image does not have to be one subjected to all of the image processing by the image processing section 22, as mentioned above) is used to retrain the second machine learning model 23a. This enables the user to acquire the current image-pickup-region information that matches the user's needs, thereby enabling to improve the accuracy of the automatic model selection mode. This enables to reduce the time and effort required for the user to operate the input section 4, thereby improving usability.

### Fifth Embodiment

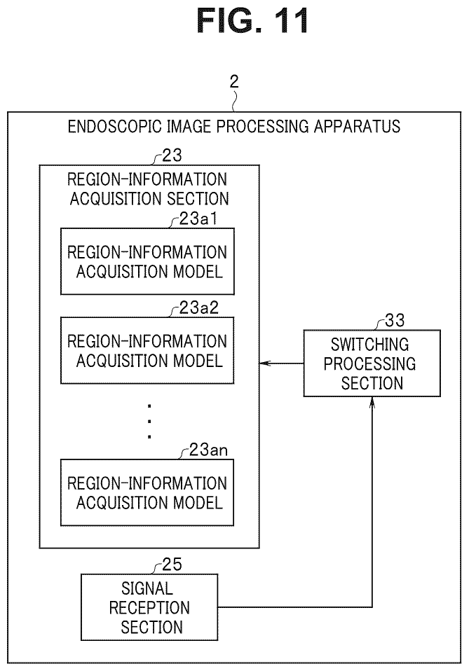

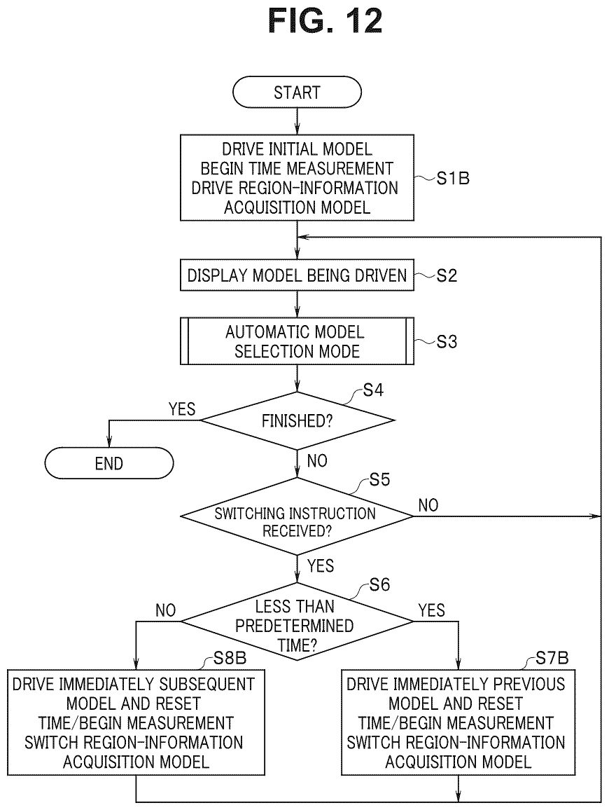

FIGS. 11 and 12 show the fifth embodiment of the present disclosure. FIG. 11 is a block diagram showing a part of a functional configuration of the endoscopic image processing apparatus 2 of the fifth embodiment. In FIG. 11, several illustrations of the configuration shown in FIG. 1 are omitted.

In the fifth embodiment, parts similar to those of the first to fourth embodiments are marked with the same reference numerals, and descriptions thereof will be omitted as appropriate. In the fifth embodiment, points different from the first to fourth embodiments will be mainly described.

In the fourth embodiment, the second machine learning model 23a is retrained; however, the present embodiment is designed such that a plurality of types of second machine learning models 23a1 to 23an are prepared in advance and are used in a switching manner.

As shown in FIG. 11, the region-information acquisition section 23 includes the plurality of types of second machine learning models 23a1 to 23an (n is an integer equal to or greater than 2) that receives input of an endoscopic image to infer the current image pickup region by the endoscope 1. The second machine learning models 23a1 to 23an are different models that may each output a different inference result (current image-pickup-region information) even when receiving input of the same endoscopic image.

For example, the second machine learning models 23a1 to 23an may be of different types of models such as DNN, CNN, R-CNN, and FCN, mentioned above. The second machine learning models 23a1 to 23an can also be models trained using different endoscopic images as training data. Furthermore, the second machine learning models 23a1 to 23an may be models using different numbers of endoscopic images as training data.

Furthermore, the endoscopic image processing apparatus 2 of the present embodiment includes a switching processing section 33 in addition to the configuration shown in FIG. 1. The processor 2a in FIG. 3 operates as the switching processing section 33 by reading and executing a processing program stored in the memory 2b. When the signal reception section 25 receives the instruction signal, the switching processing section 33 switches the machine learning models 23a1 to 23an to be used by the region-information acquisition section 23.

FIG. 12 is a flowchart showing an action of the endoscopic image processing apparatus 2 of the fifth embodiment.

When the processing in FIG. 12 begins, the selection section 26a selects and drives the initial model, the time measurement section 26c begins measuring the time since the model was selected, and the switching processing section 33 drives any preset one of the second machine learning models 23a1 to 23an (step S1B).

Thereafter, processing in steps S2 to S6 are performed, and if it is determined in step S6 that the time is less than the predetermined time, the selection section 26a selects and drives the machine learning model that it selected immediately previously, the time measurement section 26c resets the measurement time to begin the time measurement, and the switching processing section 33 switches the operating one of the second machine learning models 23a1 to 23an of the region-information acquisition section 23 to another model different from the operating one (step S7B).

If it is determined in step S6 that the time is equal to or greater than the predetermined time, the selection section 26a selects and drives the machine learning model that it is scheduled to select immediately subsequently, the time measurement section 26c resets the measurement time to begin the time measurement, and the switching processing section 33 switches the operating one of the second machine learning models 23a1 to 23an of the region-information acquisition section 23 to another model different from the operating one (step S8B).

The switching processing section 33 switches the model in this manner each time the signal reception section 25 receives the instruction signal, thereby enabling the user to set a model with a high degree of matching with the user's needs from among the second machine learning models 23a1 to 23an.

The fifth embodiment yields an effect that is almost the same as those of the first to fourth embodiments mentioned above.

According to the fifth embodiment, when the signal reception section 25 receives the instruction signal, the region-information acquisition section 23 changes the type of the second machine learning models 23a1 to 23an to be used to infer the current image pickup region. When the changed model can acquire the current image-pickup-region information that matches the user's needs, the accuracy of the automatic model selection mode can be improved. This enables to reduce the time and effort required for the user to operate the input section 4, thereby improving usability.

It is noted that the fourth to fifth embodiments described an example in which the region-information acquisition section 23 uses the machine learning models 23a, 23a1 to 23an, or the like, to acquire the current image-pickup-region information based on the endoscopic image, but are not limited to this example.

The color and texture of the plurality of regions may differ from region to region. Therefore, for example, the region-information acquisition section 23 may acquire the current image-pickup-region information by pattern matching, which compares the image of each region recorded in advance with the inputted endoscopic image, to identify a region with a high degree of similarity.

Furthermore, the region-information acquisition section 23 can identify the current image-pickup-region information using, for example, feature values such as the image compression ratio, which varies according to the frequency characteristics of the endoscopic image.

### Sixth Embodiment

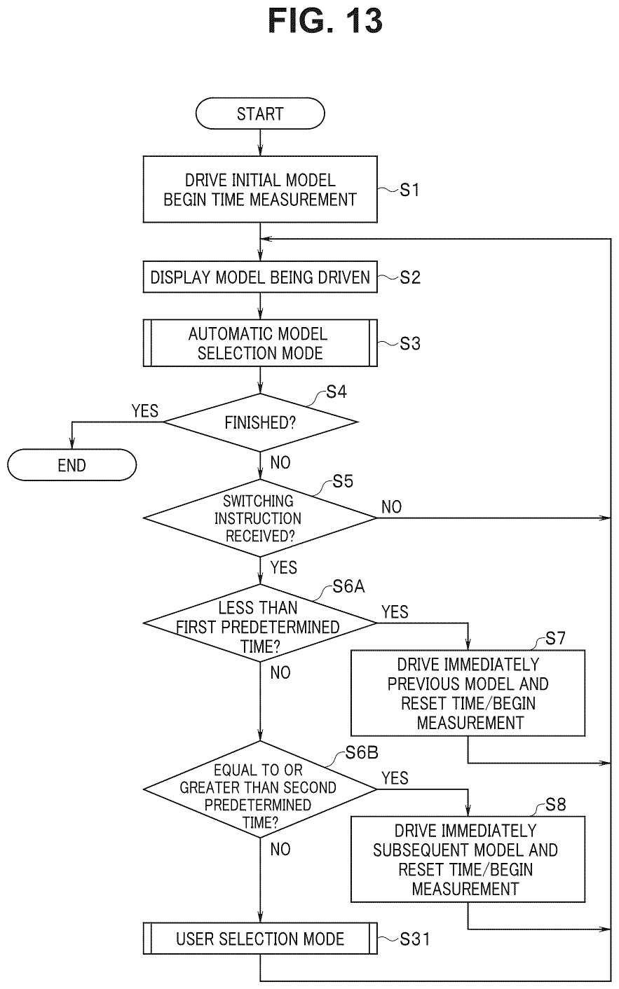

FIGS. 13 to 15 show the sixth embodiment of the present disclosure. FIG. 13 is a flowchart showing an action of an endoscopic image processing apparatus of the sixth embodiment.

In the sixth embodiment, parts similar to those of the first to fifth embodiments are marked with the same reference numerals, and descriptions thereof will be omitted as appropriate. In the sixth embodiment, points different from the first to fifth embodiments will be mainly described.

In the first to fifth embodiments, the first and second predetermined times are the same predetermined time. In contrast, in the present embodiment, the second predetermined time is longer than the first predetermined time.

When the processing in FIG. 13 begins, processing in steps S1 to S5 are performed.

If it is determined in step S5 that the instruction signal has been received, the recovery section 26d determines whether the time being measured by the time measurement section 26c (the time interval from when the selection section 26a selects the current machine learning model until the signal reception section 25 receives the instruction signal) is less than the first predetermined time (step S6A).

If it is determined here that the time is less than the first predetermined time, processing in step S7 is performed.

If it is determined in step S6A that the time is equal to or greater than the first predetermined time, the recovery section 26d determines whether or not the time being measured by the time measurement section 26c is equal to or greater than the second predetermined time (step S6B).

If it is determined here that the time is equal to or greater than the second predetermined time, processing in step S8 is performed.

If it is determined in step S6B that the time is less than the second predetermined time, that is, if the time being measured by the time measurement section 26c is equal to or greater than the first predetermined time and less than the second predetermined time, the endoscopic image processing apparatus 2 executes processing in a user selection mode (step S31).

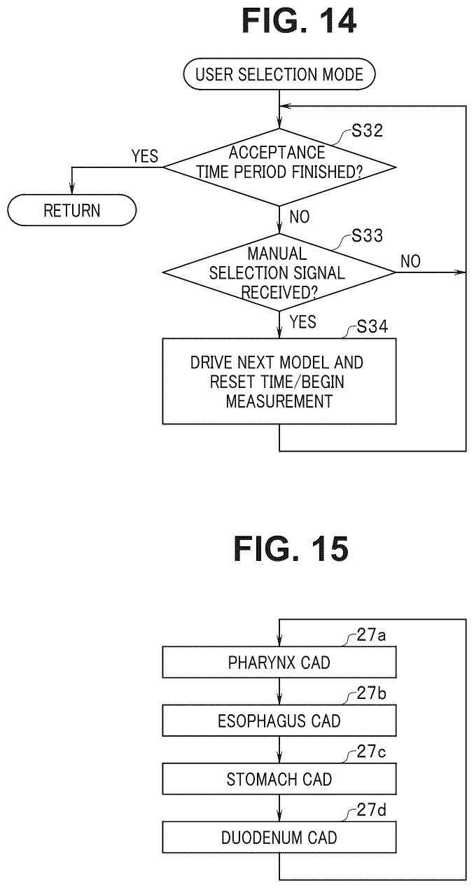

FIG. 14 is a flowchart showing the processing in the user selection mode in step S31 of FIG. 13 in the sixth embodiment.

Upon entering the user selection mode, the control section 26 determines whether or not a preset time period (acceptance time period) for accepting a user selection has finished (step S32). It is noted that the acceptance time period may finish when a fixed amount of time (fourth predetermined time) has elapsed after entering the user selection mode.

The acceptance time period can finish when either the fixed amount of time (the fourth predetermined time) has elapsed since entry into the user selection mode and the user did not perform a button operation within the fourth predetermined time, or a fixed amount of time (a fifth predetermined time) has elapsed since the user last performed a button operation. The fifth predetermined time may be shorter than the fourth predetermined time. This enables to prevent the acceptance time period from finishing when the user continues to perform the button operation even after the fourth predetermined time has elapsed.

If it is determined here that the acceptance time period has not finished, the signal reception section 25 waits to receive a manual selection signal, and the control section 26 determines whether or not the signal reception section 25 has received the manual selection signal (step S33).

That is, the signal reception section 25 receives the instruction signal as mentioned above, and further receives the manual selection signal. The manual selection signal is a signal which is generated by the input section 4 in response to the user's operation, and which selects a machine learning model from among the plurality of types of machine learning models 27a to 27d.

If it is determined here that the manual selection signal has been received, the next model is driven according to the order shown in FIG. 15, for example, and the time being measured by the time measurement section 26c is reset to begin the time measurement (step S34).

If it is determined in step S33 that no manual selection signal has been received, or if the processing in step S34 is performed, the processing returns to step S32 and the processing in step S33 is repeated until the acceptance time period finishes, and if the manual selection signal is received, the processing in step S34 is performed.

Here, FIG. 15 is a diagram showing how the machine learning models are switched in turn in the user selection mode in the sixth embodiment.

Each time it is determined in step S33 that the manual selection signal has been received, the machine learning models 27a to 27d are selected sequentially, as shown in FIG. 15.

For example, if the signal reception section 25 receives the manual selection signal when the machine learning model 27a for the pharynx is selected, the recovery section 26d causes the selection section 26a to select the machine learning model 27b for the esophagus (causes switching from the machine learning model 27a for the pharynx to the machine learning model 27b for the esophagus).

If the signal reception section 25 further receives the manual selection signal, the recovery section 26d causes the selection section 26a to select the machine learning model 27c for the stomach (causes switching from the machine learning model 27b for the esophagus to the machine learning model 27c for the stomach).

It is noted that, if the manual selection signal is received after the machine learning model 27d for the duodenum is selected, the machine learning model 27a for the pharynx is selected next.

In this manner, by the finishing of the acceptance time period, the user can select the desired model by performing, by a plurality of times, a simple operation such as pressing once a button for operating a switch of the input section 4.

If it is thereafter determined in step S32 that the acceptance time period has finished, the processing returns from the processing in FIG. 14 to that in FIG. 13. In this case, since the processing in step S31 has finished, the processing proceeds to the processing in step S2.

It is noted that, when the processing in the user selection mode shown in FIG. 14 is performed, the second machine learning model 23a in the region-information acquisition section 23 can be retrained in the same manner as described in the fourth embodiment, using the endoscopic images for several frames before and after the user selection.

In the above, the first and second predetermined times may be user-settable.

The sixth embodiment yields an effect that is almost the same as those of the first to fifth embodiments mentioned above.

According to the sixth embodiment, the user is capable of manually selecting a machine learning model in the lesion detection section 27 when the time being measured by the time measurement section 26c is equal to or greater than the first predetermined time and less than the second predetermined time. The user is also capable of selecting the desired model with an easy operation such as pressing a button by a plurality of times.

### Seventh Embodiment

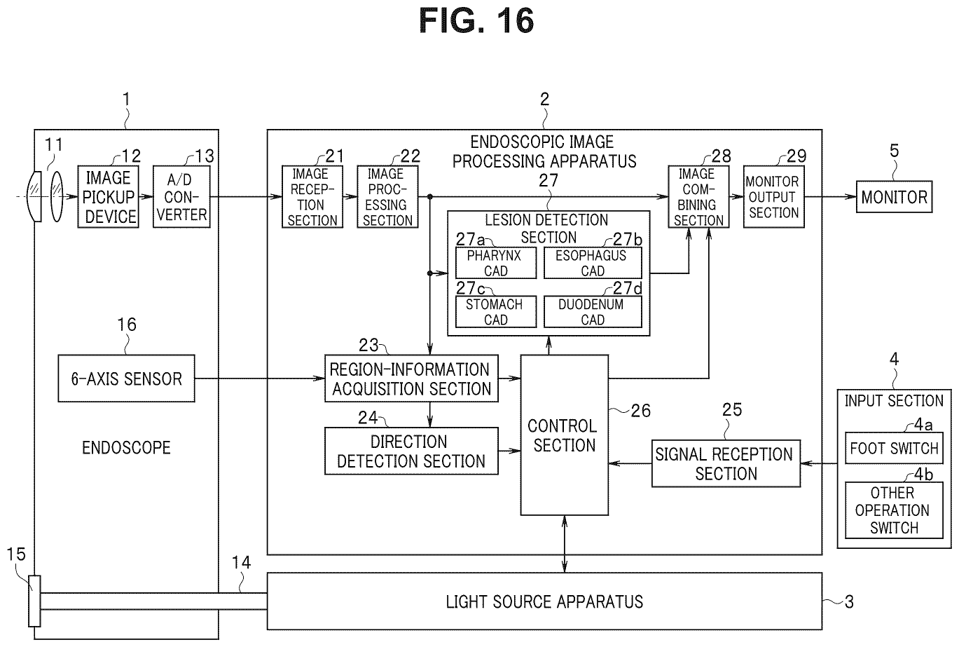

FIG. 16 is a diagram showing an example of a functional configuration of an endoscope system in the seventh embodiment of the present disclosure. In the seventh embodiment, parts similar to those of the first to sixth embodiments are marked with the same reference numerals, and descriptions thereof will be omitted as appropriate. In the seventh embodiment, points different from the first to sixth embodiments will be mainly described.

The above describes an example in which the region-information acquisition section 23 acquires the current image-pickup-region information based on the endoscopic image. In contrast, the region-information acquisition section 23 of the present embodiment is further configured to be capable of obtaining the current image-pickup-region information from a 6-axis sensor 16.

In contrast to the configuration shown in FIG. 1, the endoscope 1 of the present embodiment shown in FIG. 16 further includes the 6-axis sensor 16.

The 6-axis sensor 16 includes, for example, an x-axis acceleration sensor, a y-axis acceleration sensor, and a z-axis acceleration sensor that respectively detect accelerations in the x-, y-, and z-axis directions, which are three-dimensional orthogonal coordinate axes; and an x-axis gyroscope, a y-axis gyroscope, and a z-axis gyroscope that respectively detect angular velocities around the x-, y-, and z-axes.

The 6-axis sensor 16 is provided in the vicinity of the image pickup system including the image pickup lens 11 and the image pickup device 12, and outputs information indicating the position of the image pickup system within the subject.

In the case of the endoscopy to observe the upper gastrointestinal tract, for example, the region-information acquisition section 23 determines whether or not the endoscope has entered the oral cavity, based on the endoscopic image received from the image processing section 22.

Once having determined that the endoscope has entered the oral cavity, the region-information acquisition section 23 calculates the current position and angle of the image pickup system by computing a travel distance from a reference position and an amount of change in angle from a reference angle, based on the output from the 6-axis sensor 16, with the position and angle of the image pickup system when the endoscope entered the oral cavity as the reference.

The region-information acquisition section 23 is capable of acquiring the current image-pickup-region information, at the calculated current position of the image pickup system, and based on a direction in which the image pickup system is picking up an image and which was determined from the calculated angle.

In addition to the output from the 6-axis sensor 16, the region-information acquisition section 23 may also use the endoscopic image received from the image processing section 22 to estimate the current image pickup region, to thereby acquire the current image-pickup-region information in a more accurate manner.

The seventh embodiment yields an effect that is almost the same as those of the first to sixth embodiments mentioned above.

According to the seventh embodiment, the output of the 6-axis sensor 16 can also be used to acquire the current image-pickup-region information.

By acquiring the current image-pickup-region information using not only the output from the 6-axis sensor 16 but also the endoscopic image, the accuracy of the automatic model selection mode can be improved. This can reduce the time and effort required for the user to operate the input section 4, thereby improving usability.

### Eighth Embodiment

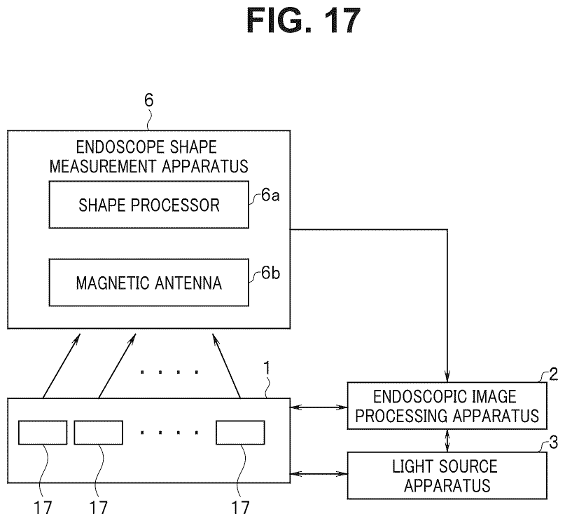

FIG. 17 is a diagram showing a part of a configuration of an endoscope system in the eighth embodiment of the present disclosure. It is noted that, in FIG. 17, illustrations of the input section 4 and the monitor 5 are omitted.

In the eighth embodiment, parts similar to those of the first to seventh embodiments are marked with the same reference numerals, and descriptions thereof will be omitted as appropriate. In the eighth embodiment, points different from the first to seventh embodiments will be mainly described.

In the seventh embodiment, the output of the 6-axis sensor 16 was used to acquire the current image-pickup-region information. In contrast, the eighth embodiment is a configuration in which an external device that utilizes magnetism, for example, is used to acquire the current image-pickup-region information.

The endoscope 1 includes a plurality of magnetic generators 17 arranged, for example, along the axis direction of the insertion portion. The magnetic generators 17 are instruments that generate magnetism, such as magnets and coils. It is noted that the positional relationship along the axis direction of the insertion portion between the plurality of magnetic generators 17 and the image pickup system provided at the distal end portion of the insertion portion of the endoscope 1 is known based on the design and other factors.

In addition to the configuration shown in FIG. 1, the endoscope system further includes an endoscope shape measurement apparatus 6. The endoscope shape measurement apparatus 6 includes a shape processor 6a and a magnetic antenna 6b.

The magnetic antenna 6b detects the magnetism generated by each of the plurality of magnetic generators 17.

The shape processor 6a calculates a position and/or shape of the insertion portion of the endoscope 1 based on a detection result of the magnetic antenna 6b.

Specifically, the shape processor 6a calculates respective positions of the plurality of magnetic generators 17 arranged in the insertion portion of the endoscope 1. Once the positions of the plurality of magnetic generators 17 are calculated, a bending shape (three-dimensional shape) of the insertion portion, the position of the image pickup system provided at the distal end portion of the insertion portion, and the like can also be calculated. Once the bending shape of the insertion portion is known, the direction in which the image pickup system is oriented can also be calculated.

The endoscope shape measurement apparatus 6 is connected to the endoscopic image processing apparatus 2. The endoscopic image processing apparatus 2 acquires information of the position and direction of the image pickup system from the endoscope shape measurement apparatus 6, and the region-information acquisition section 23 identifies the current image pickup region.

It is noted that, although the above described is an example of using an external device that utilizes magnetism, no limitation is placed thereon, and external devices that utilize electromagnetic waves or other physical quantities may also be used.

The eighth embodiment yields an effect that is almost the same as those of the first to seventh embodiments mentioned above.

According to the eighth embodiment, when the endoscope system includes the endoscope shape measurement apparatus 6, the information of the shape of the endoscope 1 measured by the endoscope shape measurement apparatus 6 can be utilized to acquire the current image-pickup-region information.

It may be configured to acquire the current image-pickup-region information in a more accurate manner by combining, as necessary, the method for acquiring the current image-pickup-region information based on the endoscopic image described in the first to sixth embodiments, the method for acquiring the current image-pickup-region information using the output of the 6-axis sensor 16 described in the seventh embodiment, and the method for acquiring the current image-pickup-region information utilizing a measurement result of the endoscope shape measurement apparatus 6 in the present embodiment.

### Ninth Embodiment

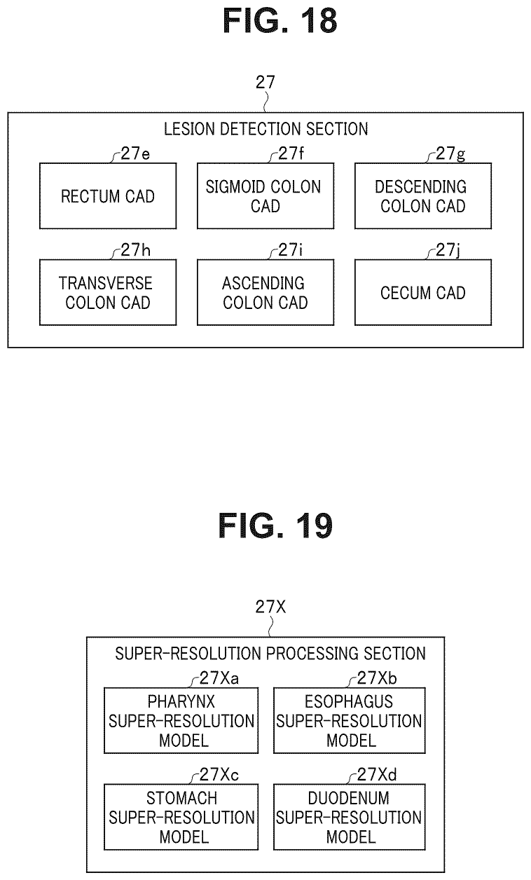

FIG. 18 is a block diagram showing an example of a configuration of the lesion detection section 27 when an endoscopic observation of the lower gastrointestinal tract is performed in the ninth embodiment of the present disclosure. In the ninth embodiment, parts similar to those of the first to eighth embodiments are marked with the same reference numerals, and descriptions thereof will be omitted as appropriate. In the ninth embodiment, points different from the first to eighth embodiments will be mainly described.

The above described is an example of mainly performing the endoscopic observation of the upper gastrointestinal tract, but the present embodiment describes an example of performing the endoscopic observation of the lower gastrointestinal tract.

The lower gastrointestinal tract includes, as the plurality of regions, organs of, e.g., the rectum, sigmoid colon, descending colon, transverse colon, ascending colon, and cecum, that are arranged in order in the subject.

The lesion detection section 27 includes, as the plurality of types of machine learning models, a machine learning model for the rectum (rectum CAD) 27e, a machine learning model for the sigmoid colon (sigmoid colon CAD) 27f, a machine learning model for the descending colon (descending colon CAD) 27g, a machine learning model for the transverse colon (transverse colon CAD) 27h, a machine learning model for the ascending colon (ascending colon CAD) 27i, and a machine learning model for the cecum (cecum CAD) 27j, which are respectively adapted to the organs of the rectum, sigmoid colon, descending colon, transverse colon, ascending colon, and cecum.

The machine learning model 27e for the rectum (rectum CAD) is a trained model that is trained for CAD using images of the rectum as training data. The machine learning model 27f for the sigmoid colon (sigmoid colon CAD) is a trained model that is trained for CAD using images of the sigmoid colon as training data.

The machine learning model 27g for the descending colon (descending colon CAD) is a trained model that is trained for CAD using images of the descending colon as training data.

The machine learning model 27h for the transverse colon (transverse colon CAD) is a trained model that is trained for CAD using images of the transverse colon as training data. The machine learning model 27i for ascending colon (ascending colon CAD) is a trained model that is trained for CAD using images of the ascending colon as training data. The machine learning model 27j for the cecum (cecum CAD) is a trained model that is trained for CAD using images of the cecum as training data.

It is noted that, when the region-information acquisition section 23 uses second machine learning models for respective regions, it is sufficient to use second machine learning models similarly adapted to the organs of the rectum, sigmoid colon, descending colon, transverse colon, ascending colon, and cecum, respectively.

Also in the present embodiment, the predetermined time (hereinafter including first predetermined time and second predetermined time) when the selection section 26a selects a machine learning model adapted to the first region among the plurality of regions may be made different from the predetermined time when the selection section 26a selects a machine learning model adapted to another region than the first region. For example, a specific example of making the predetermined time different is as follows.

The predetermined time when the selection section 26a selects the machine learning model 27g for the descending colon is longer than the predetermined time when selecting the machine learning model 27e for the rectum, longer than the predetermined time when selecting the machine learning model 27f for the sigmoid colon, and longer than the predetermined time when selecting the machine learning model 27j for the cecum.

The predetermined time when the selection section 26a selects the machine learning model 27h for the transverse colon is longer than the predetermined time when selecting the machine learning model 27e for the rectum, longer than the predetermined time when selecting the machine learning model 27f for the sigmoid colon, and longer than the predetermined time when selecting the machine learning model 27j for the cecum.

The predetermined time when the selection section 26a selects the machine learning model 27i for the ascending colon is longer than the predetermined time when selecting the machine learning model 27e for the rectum, longer than the predetermined time when selecting the machine learning model 27f for the sigmoid colon, and longer than the predetermined time when selecting the machine learning model 27j for the cecum.

It is noted that, as mentioned above, a plurality of models corresponding to the types of light sources may be provided for the same region.

The ninth embodiment yields an effect that is almost the same as those of the first to eighth embodiments mentioned above, even when the endoscopic observation of the Lower gastrointestinal tract is performed.

### Tenth Embodiment

FIG. 19 is a block diagram showing an example of a configuration in which a super-resolution processing section 27X is provided in place of the lesion detection section 27 in a tenth embodiment of the present disclosure. In the tenth embodiment, parts similar to those of the first to ninth embodiments are marked with the same reference numerals, and descriptions thereof will be omitted as appropriate. In the tenth embodiment, points different from the first to ninth embodiments will be mainly described.

The above-mentioned embodiments each described an example in which the lesion detection section 27 includes a machine learning model for CAD, but the machine learning model is not limited thereto.

In the present embodiment, a machine learning model to perform super resolution of images is provided. In other words, the endoscopic image processing apparatus 2 includes the super-resolution processing section 27X in place of the lesion detection section 27.

As shown in FIG. 19, the super-resolution processing section 27X includes, as the plurality of types of machine learning models, a machine learning model 27Xa for the pharynx (pharynx super-resolution model), a machine learning model 27Xb for the esophagus (esophagus super-resolution model), a machine learning model 27Xc for the stomach (stomach super-resolution model), and a machine learning model 27Xd for the duodenum (duodenum super-resolution model), which are respectively adapted to the organs of the pharynx, esophagus, stomach, and duodenum.

The machine learning model 27Xa for the pharynx is a trained model that is trained for super resolution using images of the pharynx as training data. Upon receiving input of an endoscopic image of the pharynx sent from the image processing section 22, the machine learning model 27Xa for the pharynx performs inference to increase the resolution of the endoscopic image of the pharynx, and outputs an endoscopic image subjected to super-resolution processing after the inference.

The machine learning model 27Xb for the esophagus is a trained model that is trained for super resolution using images of the esophagus as training data. Upon receiving input of an endoscopic image of the esophagus sent from the image processing section 22, the machine learning model 27Xb for the esophagus performs inference to increase the resolution of the endoscopic image of the esophagus and outputs an endoscopic image subjected to super-resolution processing after the inference.

The machine learning model 27Xc for the stomach is a trained model that is trained for super resolution using images of the stomach as training data. Upon receiving input of an endoscopic image of the stomach sent from the image processing section 22, the machine learning model 27Xc for the stomach performs inference to increase the resolution of the endoscopic image of the stomach and outputs an endoscopic image subjected to super-resolution processing after the inference.

The machine learning model 27Xd for the duodenum is a trained model that is trained for super resolution using images of the duodenum as training data. Upon receiving input of an endoscopic image of the duodenum sent from the image processing section 22, the machine learning model 27Xd for the duodenum performs inference to increase the resolution of the endoscopic image of the duodenum and outputs an endoscopic image subjected to super-resolution processing after the inference.

It is noted that, in the example of performing the endoscopic observation of the lower gastrointestinal tract, it is sufficient to use, for example, super-resolution models that are respectively adapted to the organs of the rectum, sigmoid colon, descending colon, transverse colon, ascending colon, and cecum.

According to the tenth embodiment, when an automatically selected super-resolution machine learning model does not match the user's needs, the user can select a super-resolution machine learning model that matches the user's needs with a simple operation such as pressing a button once.

The user does not need to perform complex operations to select a machine learning model. This enables to suppress a decrease in the labor-saving effectiveness (the effect of saving user's labor) of the endoscopic image processing apparatus 2 having the function of automatically selecting a super-resolution machine learning model. This enables the user to concentrate on operating the endoscope 1 and checking the display on the monitor 5, thereby improving the efficiency of endoscopy. In addition, the user is enabled to observe lesions and other objects in greater detail by viewing the endoscopic image subjected to super-resolution processing.

It is noted that the above described an example in which the endoscopic image processing apparatus 2 includes the super-resolution processing section 27X in place of the lesion detection section 27, but the endoscopic image processing apparatus 2 may include both the lesion detection section 27 and the super-resolution processing section 27X. In this case, the tenth embodiment further yields an effect that is almost the same as those of the first to ninth embodiments mentioned above.

As mentioned above, a plurality of super-resolution models corresponding to the types of light sources may be provided for the same region.

In the above, machine learning models for CAD and machine learning models for super resolution were described as automatically selected machine learning models, but no limitation is placed thereon, and other machine learning models can also be utilized.

It is noted that, the above mainly described a case in which the present disclosure is an endoscopic image processing apparatus, but the disclosure is not limited thereto. For example, the present disclosure may be a method for operating the endoscopic image processing apparatus, or may be an endoscopic image processing method that performs processing similar to that performed by the endoscopic image processing apparatus. The present disclosure may also be a computer program (endoscopic image processing program) for causing a computer to perform processing similar to that performed by the endoscopic image processing apparatus. Furthermore, the present disclosure may be a computer readable non-transitory recording medium, or the like, which records the computer program (endoscopic image processing program).

Here, some examples of recording media that store computer program products include portable recording media such as flexible disks, compact disc read only memory (CD-ROM), digital versatile discs (DVD), and other recording media such as hard disk drives (HDD), solid state drives (SSD), and the like. Flexible disks, CD-ROMs, DVDs, and hard disks are examples of nonvolatile storage media. What is stored in the recording medium is not limited to the entire computer program, but may be a part thereof. The entire computer program or a part thereof may also be distributed or provided via a communication network. By installing a computer program on a computer from a recording medium, or by downloading and installing the computer program on a computer via a communication network, the user enables the computer to read the computer program and perform all or a part of the operations, thereby enabling to perform the operation of the endoscopic image processing apparatus mentioned above.

Furthermore, the present disclosure is not limited to the above-mentioned embodiments as they are. The present disclosure is capable of being embodied by varying constituent elements in the implementation stage within a scope not departing from the gist of the disclosure. In addition, the plurality of constituent elements disclosed in the above embodiments are capable of being combined as appropriate to form various aspects of the disclosure. For example, some constituent elements may be deleted from all the constituent elements disclosed in the embodiments. Furthermore, constituent elements of different embodiments may be combined as appropriate. Thus, it is needless to say that various variations and applications are possible within a scope not departing from the gist of the disclosure.

What is claimed is:

1. An endoscopic image processing apparatus including one or more processors,
the one or more processors being configured to:
select a machine learning model from among a plurality of types of machine learning models according to a region whose image is being picked up by an endoscope;
generate notification information of a type of the machine learning model selected;
receive an instruction signal for switching the machine learning model, the instruction signal being generated in response to a user's operation;
measure a time interval from a selection of the machine learning model to a reception of the instruction signal;
when the time interval is less than a first predetermined time, select a machine learning model selected immediately previously; and
when the time interval is equal to or greater than a second predetermined time that is the same as or longer than the first predetermined time, select a machine learning model scheduled to be selected immediately subsequently.

2. The endoscopic image processing apparatus according to claim 1, wherein the one or more processors acquire current image-pickup-region information, and select the machine learning model from among the plurality of types of machine learning models according to the current image-pickup-region information acquired.

3. The endoscopic image processing apparatus according to claim 2, wherein
the one or more processors detect a direction of travel of the endoscope, select the machine learning model from among the plurality of types of machine learning models based on a switching order defined according to an arrangement of a plurality of regions in a subject, and to the direction of travel detected, and schedule a machine learning model to be selected immediately subsequently.

4. The endoscopic image processing apparatus according to claim 1, wherein the one or more processors prohibit the selection of the machine learning model according to the region whose image is being picked up, for a third predetermined time after the reception of the instruction signal.

5. The endoscopic image processing apparatus according to claim 1, wherein the first predetermined time and the second predetermined time are the same.

6. The endoscopic image processing apparatus according to claim 1, wherein
the second predetermined time is longer than the first predetermined time, and
when the time interval is equal to or greater than the first predetermined time and less than the second predetermined time, the one or more processors wait to receive a manual selection signal for selecting the machine learning model from among the plurality of types of machine learning models, the manual selection signal being generated in response to the user's operation, and upon receiving the manual selection signal, the one or more processors select a machine learning model indicated by the manual selection signal.

7. The endoscopic image processing apparatus according to claim 1, wherein
the first predetermined time when a machine learning model adapted to a first region among a plurality of regions in a subject is selected differs, in length of time, from the first predetermined time when a machine learning model adapted to another region than the first region is selected, and
the second predetermined time when the machine learning model adapted to the first region among the plurality of regions is selected differs, in the length of time, from the second predetermined time when the machine learning model adapted to another region than the first region is selected.

8. The endoscopic image processing apparatus according to claim 7, wherein
the plurality of regions are organs including a pharynx, an esophagus, a stomach, and a duodenum,
the plurality of types of machine learning models include a machine learning model for the pharynx, a machine learning model for the esophagus, a machine learning model for the stomach, and a machine learning model for the duodenum,
the first and second predetermined times when the machine learning model for the stomach is selected are longer than the first and second predetermined times when the machine learning model for the pharynx is selected, and longer than the first and second predetermined times when the machine learning model for the duodenum is selected, and
the first and second predetermined times when the machine learning model for the esophagus is selected are longer than the first and second predetermined times when the machine learning model for the pharynx is selected, and longer than the first and second predetermined times when the machine learning model for the duodenum is selected.

9. The endoscopic image processing apparatus according to claim 7, wherein
the plurality of regions are organs including a rectum, a sigmoid colon, a descending colon, a transverse colon, an ascending colon, and a cecum,
the plurality of types of machine learning models include a machine learning model for the rectum, a machine learning model for the sigmoid colon, a machine learning model for the descending colon, a machine learning model for the transverse colon, a machine learning model for the ascending colon, and a machine learning model for the cecum,
the first and second predetermined times when the machine learning model for the descending colon is selected are longer than the first and second predetermined times when the machine learning model for the rectum is selected, longer than the first and second predetermined times when the machine learning model for the sigmoid colon is selected, and longer than the first and second predetermined times when the machine learning model for the cecum is selected,
the first and second predetermined times when the machine learning model for the transverse colon is selected are longer than the first and second predetermined times when the machine learning model for the rectum is selected, longer than the first and second predetermined times when the machine learning model for the sigmoid colon is selected, and longer than the first and second predetermined times when the machine learning model for the cecum is selected, and
the first and second predetermined times when the machine learning model for the ascending colon is selected is longer than the first and second predetermined times when the machine learning model for the rectum is selected, longer than the first and second predetermined times when the machine learning model for the sigmoid colon is selected, and longer than the first and second predetermined times when the machine learning model for the cecum is selected.

10. The endoscopic image processing apparatus according to claim 1, wherein
the instruction signal includes a first instruction signal generated from a foot switch, and a second instruction signal generated from an operation switch other than the foot switch, and
the first and second predetermined times that are set for a case where the first instruction signal is received are longer than the first and second predetermined times that are set for a case where the second instruction signal is received.

11. The endoscopic image processing apparatus according to claim 1, wherein the first and second predetermined times are settable by the user.

12. The endoscopic image processing apparatus according to claim 2, wherein
the one or more processors receive an endoscopic image acquired by the endoscope picking up an image of one or more of a plurality of regions in a subject,
the machine learning model receives input of the endoscopic image to perform inference, and
the one or more processors output an image to a monitor, the image including the endoscopic image and an inference result from the machine learning model.

13. The endoscopic image processing apparatus according to claim 12, wherein, when receiving the instruction signal, the one or more processors use the endoscopic image pertaining to the image that is outputted to the monitor when receiving the instruction signal, to retrain a second machine learning model that receives input of the endoscopic image to infer a current image pickup region.

14. The endoscopic image processing apparatus according to claim 12, wherein, when receiving the instruction signal, the one or more processors switch a type of a second machine learning model used by the one or more processors, among a plurality of types of second machine learning models that each receive input of the endoscopic image to infer a current image pickup region.

15. A method for operating an endoscopic image processing apparatus including one or more processors, the one or more processors being configured to:
select a machine learning model from among a plurality of types of machine learning models according to a region whose image is being picked up by an endoscope;
generate notification information of a type of the machine learning model selected;
receive an instruction signal for switching the machine learning model, the instruction signal being generated in response to a user's operation;
measure a time interval from a selection of the machine learning model to a reception of the instruction signal;
when the time interval is less than a first predetermined time, select a machine learning model selected immediately previously; and
when the time interval is equal to or greater than a second predetermined time that is the same as or longer than the first predetermined time, select a machine learning model scheduled to be selected immediately subsequently.

16. A nonvolatile storage medium storing an endoscopic image processing program, the program causing one or more computers to perform a process comprising:
selecting a machine learning model from among a plurality of types of machine learning models according to a region whose image is being picked up by an endoscope;
generating notification information of a type of the machine learning model selected;
receiving an instruction signal for switching the machine learning model, the instruction signal being generated in response to a user's operation;
measuring a time interval from a selection of the machine learning model to a reception of the instruction signal;
when the time interval is less than a first predetermined time, selecting a machine learning model selected immediately previously; and
when the time interval is equal to or greater than a second predetermined time that is the same as or longer than the first predetermined time, selecting a machine learning model scheduled to be selected immediately subsequently.
